(19) United States
(12) Patent Application Publication
(10) Pub. No.: US 2016/0324451 A1
(43) Pub. Date: Nov. 10, 2016

(54) Use google's smart contact lens for measuring glucose levels in tears to enhance executive and non-executive functions in humans

(71) Applicant: Michael Roy Young, Danville, CA (US)

(72) Inventor: Michael Roy Young, Danville, CA (US)

(21) Appl. No.: 14/545,441

(22) Filed: May 6, 2015

(51) Int. Cl. A61B 5/145; A61B 5/00

(52) U.S. Cl. CPC A61B 5/14532; A61B 5/6821

(57) ABSTRACT

Google's smart contact lens will be used by non-diabetics to benefit from the measurement of the glucose content in the tears of the wearer as a representation of the glucose content in the blood. With this data, non-diabetics will be able to modify their diet as necessary to stabilize and optimize their blood glucose content. This will result in their avoiding the normal sharp peaks and valleys in their blood glucose levels that occur as meals are digested and instead enable them to maintain their blood glucose content at a steady level. Instead of the brain experiencing low flows of energy that causes impairment in its ability to carry out mental functions this steady flow of energy to their brain will result in enhancement of a number of executive and non-executive functions including, but not limited to, memory, decision making, reading skills, attentiveness, self-control, verbal fluency, auditory and visual reaction time and psychomotor skills.

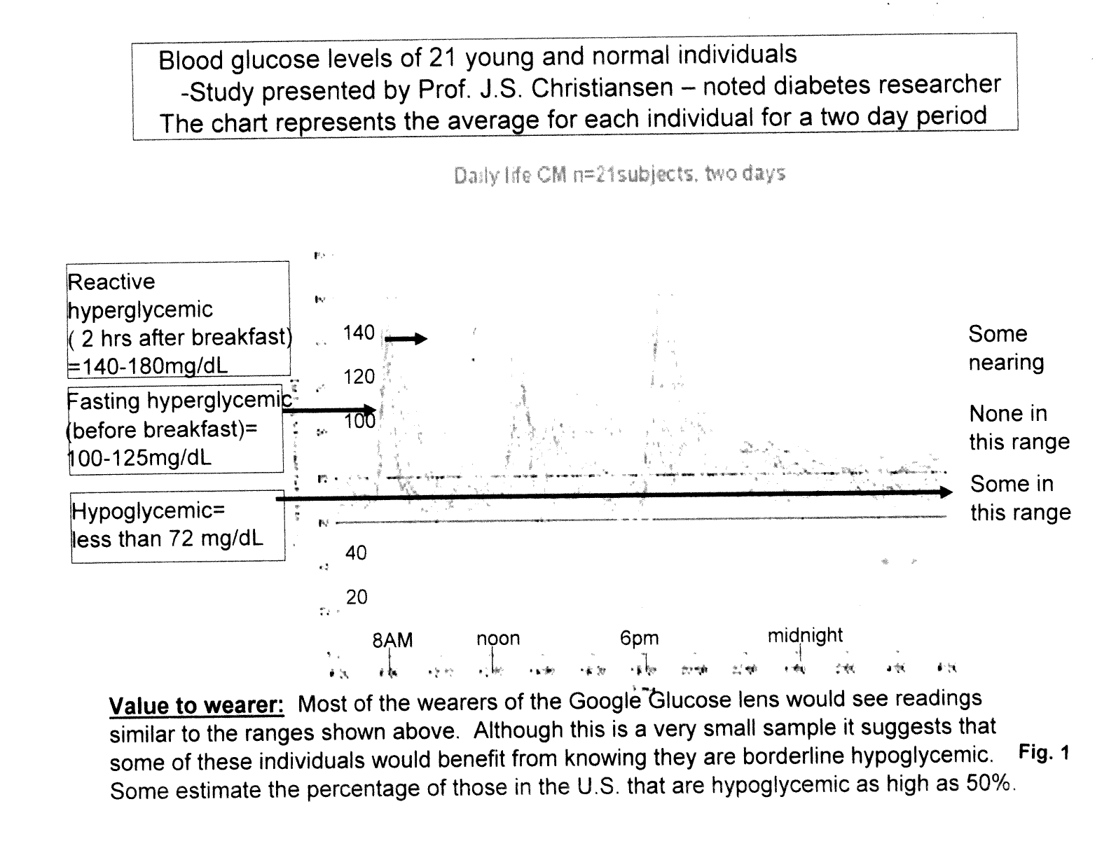

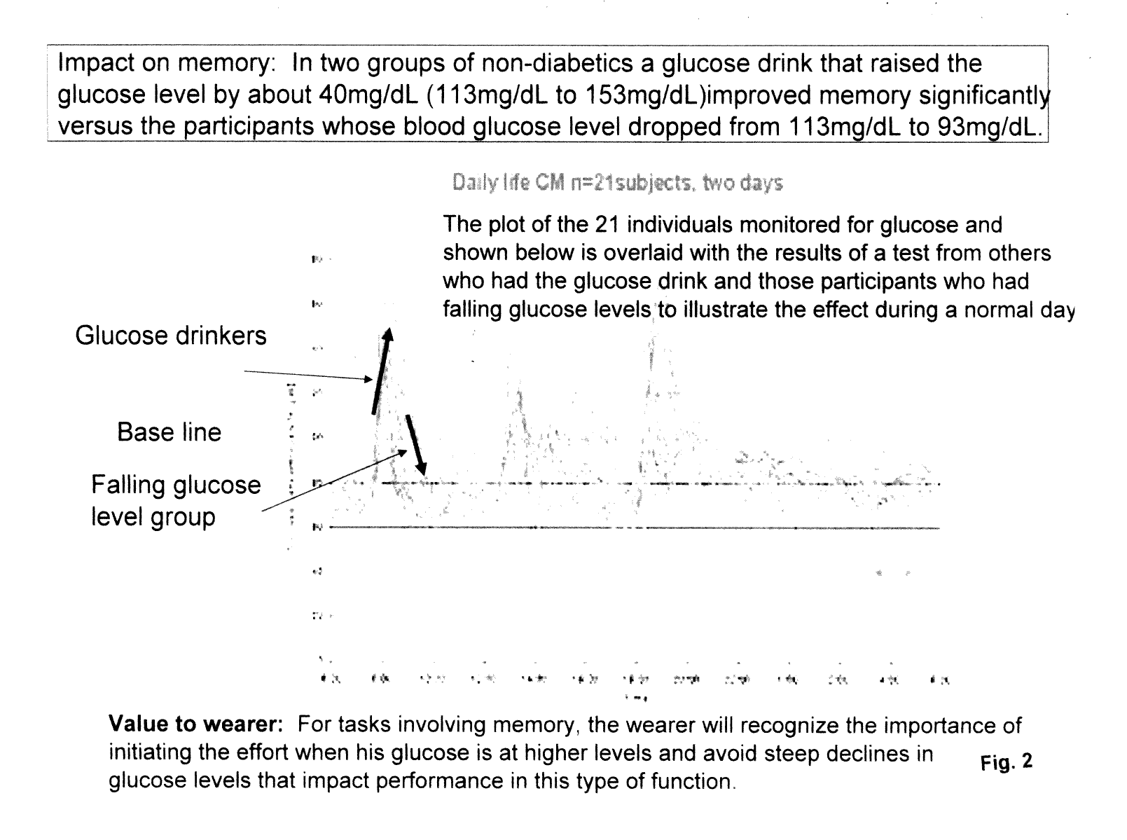

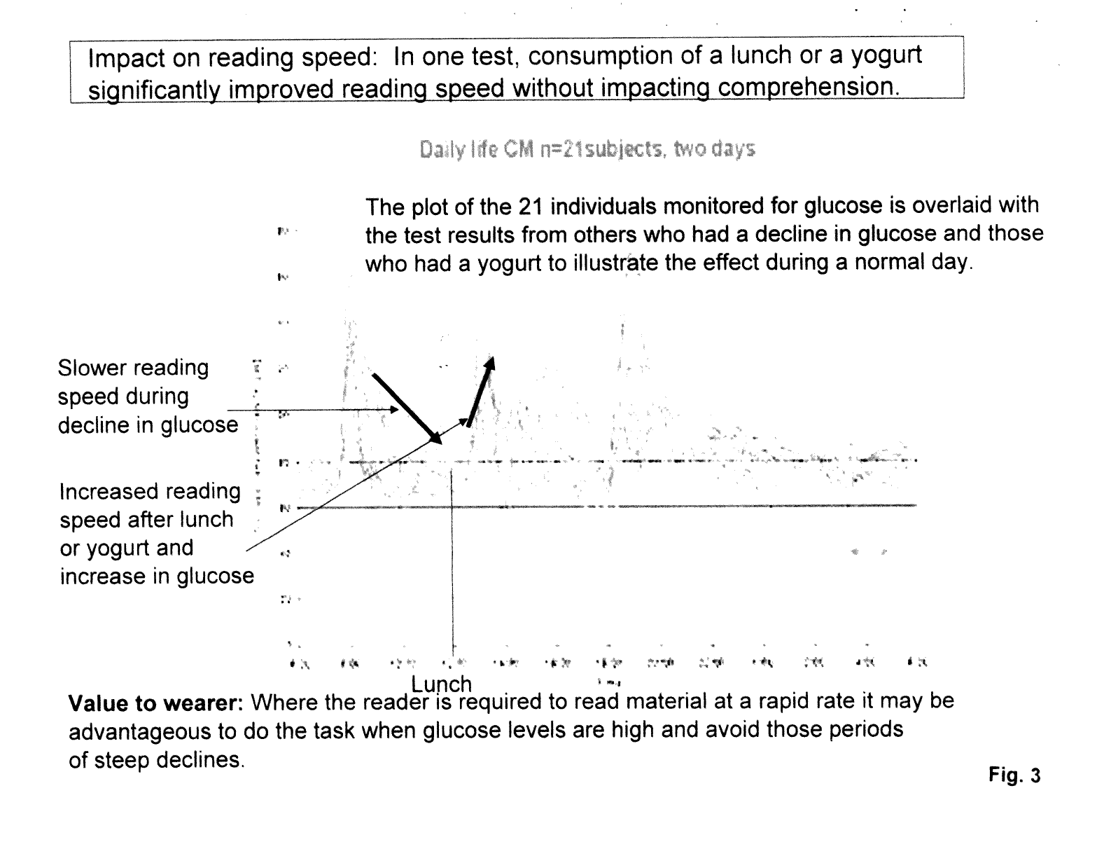

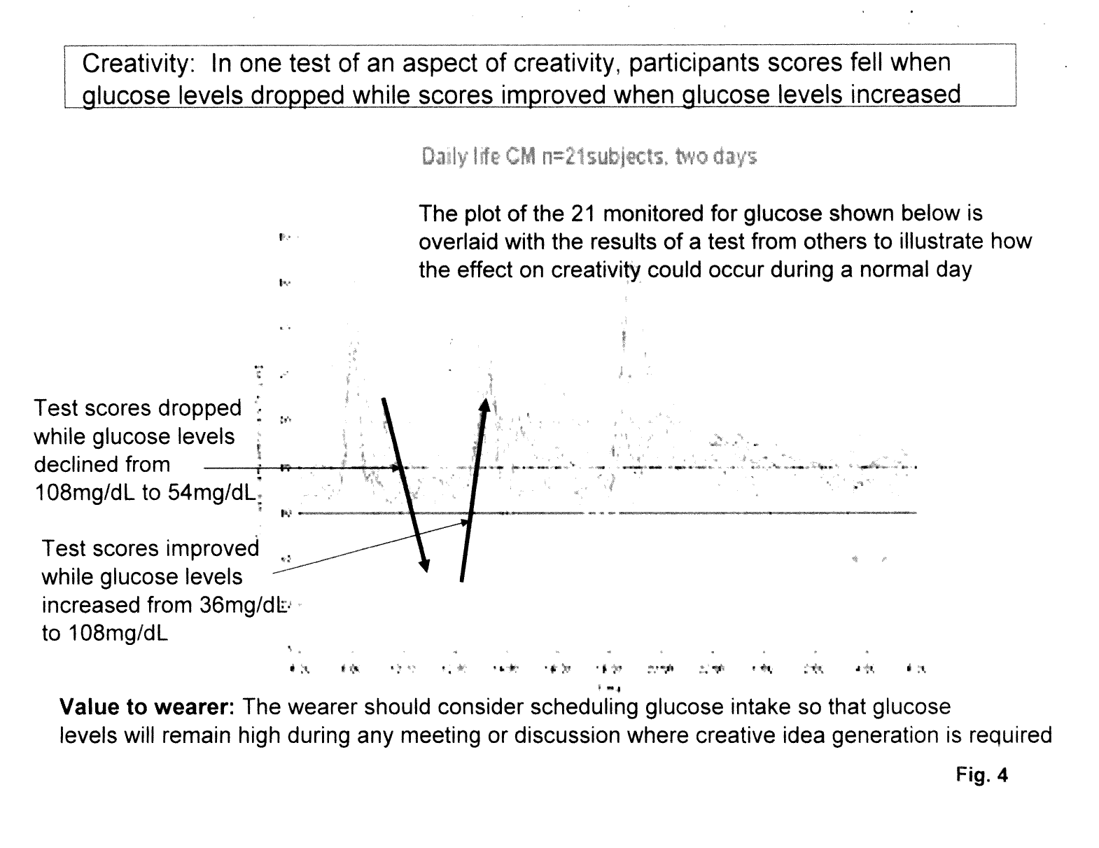

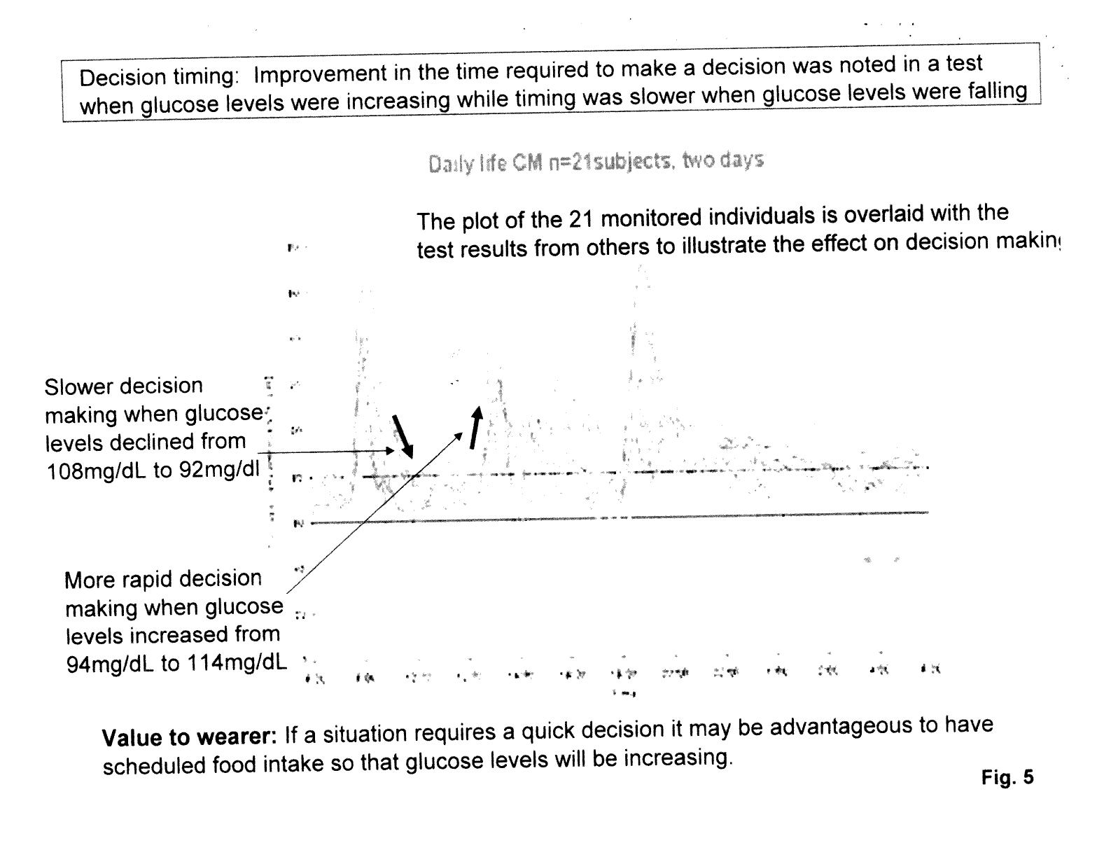

### CROSS REFERENCE OF THE INVENTION

This application claims the benefit of U.S. Provisional Patent No. 61/998,626 filed Jul. 3, 2014

### TECHNICAL FIELD

By measuring the flow of glucose to the brain the wearer can regulate his/her diet to keep the glucose at an optimum level. This will result in the wearer avoiding the peaks and valleys of blood glucose levels that are normally experienced. Tests have been conducted that point to the impairment of certain brain functions when the flow of energy to the brain drops to low levels. The measurement of the flow of energy to the brain is the key to determining what foods and drinks to consume and when to eat to ensure that low glucose levels are avoided. By avoiding these levels several critical brain functions including self-control, memory, decision making, judgment, attention and visual skills will all be enhanced.

### BACKGROUND OF THE INVENTION

### Background

Scientists have studied various ways to measure blood glucose levels including the glucose level in sweat, breath and saliva. On Sep. 27, 2012 a U.S. Patent Application number 20120245444 was granted to Brian Otis, Yu-Te Liao, Babak Amirparviz and Huanfen Yao for the invention of a ‘Wireless Powered Contact lens with Glucose Sensor’.

On Jan. 16, 2014 Google announced the introduction of their smart contact lens project. As noted in their announcement, the intent of lens is to measure the glucose level in the tears of the wearer as a means of providing people suffering from diabetes a means of continuously monitoring their blood sugar level without the need to draw blood. Goggle's studies suggest that the glucose level in the tears provide a reasonably accurate method of measuring the glucose level in the blood. The invention of Google's smart contact lens capable of measuring the glucose content of the tears of the wearer will provide a visual display on a smart phone, tablet or computer of the continuous level of the glucose that is representative of the wearer's blood glucose level.

In July of 2014, Google announced a partnership with Alcon, a subsidiary of Novartis to further the technology for eventual commercialization. The stated purpose of both Google and Alcon is to provide diabetics with the ability to better maintain their blood sugar level and to avoid the need to draw blood several times a day. This will be an important use of the device but there is a far more valuable use of the lens by providing all of mankind with the ability to measure the flow of glucose to the brain, the source of the brain's energy.

### SUMMARY OF INVENTION

### Technical Problem

Glucose is derived from carbohydrates and is a sugar that provides the energy that the body and brain need to function. The brain consumes nearly twenty percent of the glucose that the body produces, more than any other organ. There is a limited amount of glucose stored in the brain. It cannot continue to function if it is deprived of glucose for a period as short as ten minutes. In a study conducted by the Department of Psychology in the University of Wales Swansea in 1994 it was noted that ‘There is increasing evidence that the provision of blood glucose influences memory.’ The results of the tests in the study resulted in the conclusion that ‘the present data suggests that in normal healthy individuals the provision of blood glucose influences the performance of some non-memory tasks.’

The normal range of blood glucose in non-diabetics varies widely: A paper, published in the ‘Journal of Diabetics Science and Technology From Basic Science to Clinical Practice’ dated September 2007 titled ‘Continuous Glucose Profiles in Healthy Subjects under Everyday Life Conditions and After Different Meals’ provided data on the variation in glucose levels during a 24 hour period where the non-diabetic subjects ate whatever they chose to eat at home at designated times. Each subject was fitted with two continuous glucose monitors and the resulting data is the average of the two readings. The results of the study were presented by Professor J. S. Christiansen at the annual meeting of the European Association for the Study of Diabetes on Sep. 13, 2006. The data indicated that these normal individuals had blood glucose levels that approached both hyperglycemic and hypoglycemic levels.

The normal diet results in relatively sharp peaks and valleys in blood glucose levels. The normal diet results in relatively sharp peaks and valleys in blood glucose levels. Studies on non-diabetics have shown that they experience elevated blood glucose levels shortly after their daily meals that approach the hyperglycemic range. Blood glucose levels in this range over an extended period can result in damage to the kidneys, nerves, eyes or legs and feet. Impaired memory may also result. The same studies also shown that non-diabetics experience low blood sugar levels that can approach the hypoglycemic level as their blood sugar level drops an hour or more after a meal. Depending on the individual and the level of blood glucose, decreasing blood sugar levels can result in loss of memory, slower reading skills and slower ability to make decisions. In addition, again depending on the individual, blood glucose levels, reaching the hypoglycemic level can result in reduced self-control, reduced attentiveness, a reduction in mental flexibility and a reduction in auditory and visual reaction time. Without the ability to continually measure blood glucose levels the individual experiences some level of impairment in these critical brain functions without even knowing how or to what extent they have been impacted.

Lack of measurement and control of blood glucose levels impacts memory: NASA prepared a review paper titled ‘The Effects of Blood Glucose Levels on Cognitive Performance: A Review of the Literature’. This paper, conducted by researchers at San Jose State University and Ames Research Center, dated June 2007, provided a summary of the research done dating back 20 years on the effects of differing glucose levels on executive and non-executive functions. The conclusions in the NASA paper attest to the benefit of glucose, in a drink or carbohydrate snack, in terms of significantly enhancing memory performance. The ability to recall and the speed of performance in memory testing on non-diabetic adults have been positively related to blood glucose levels. One of the studies was quoted in the paper as noting that “The enhancing effects of glucose on memory (e.g. better retention, reduced-forgetting) have been reported well within normal blood glucose levels.”

Blood glucose levels impact reading skills: The NASA review also noted the studies that documented the benefits of increased glucose levels on reading speed and impaired performance at low glucose levels for non-diabetics.

Blood glucose levels impact attentiveness: In addition, the NASA review noted a 2000 study that found reaction time in both diabetics and non-diabetics was affected at low glucose levels. Other studies concluded that ‘visual and auditory selective attention and visual attentional switching were affected in non-diabetics at low blood glucose levels.

Blood glucose levels impact psychomotor tasks: The NASA summary comments that ‘Reaction time performance has been affected in specific psychomotor tasks at low blood glucose levels, in both diabetic and non-diabetic individuals.’ These effects have also been demonstrated on more difficult psychomotor tasks (e.g., reaction time, fine motor activity and hand eye coordination). The summary concludes that ‘It is clear that there exists a continuum of degradation of performance on specific tasks at certain glucose levels.’

Blood glucose levels impact verbal and auditory skills: The NASA summary noted that ‘Blood glucose levels from a glucose drink have affected verbal fluency performance in non-diabetics’ The summary also noted that ‘Blood glucose levels have affected performance of basic auditory processing in non-diabetic individuals’.

Blood glucose levels impact decision making: A study conducted at the University of South Dakota in 2010 and summarized in a paper prepared by the Association for Psychological Science titled ‘Sweet Future: Fluctuating Blood Glucose Levels May Affect Decision Making’ commented that ‘Studies have indicated that there may be a link between blood glucose levels (our body's energy) and thinking.’ The study found that the non-diabetic test subjects were more likely to be more future oriented. The paper commented that ‘These findings are suggestive of an adaptive mechanism linking decision to metabolic cues, such as blood sugar levels. In another study, healthy non-diabetic college students showed significant impairment in tests to measure aspects of strategic thinking when their blood glucose dropped to low levels.

Blood glucose levels impact self-control: A study conducted by Professor Brad Bushman at Ohio State University in 2012 tested 21 married couples concluded that low blood glucose levels may make spouses more angry and more likely to be aggressive towards one another when they experienced lower levels of glucose in their blood. In summarizing the results of the study Professor Bushman is quoted as saying “The study shows how one simple often over looked factor—caused by low levels of blood glucose—may play a role in marital arguments, confrontations and possibly even some domestic violence.” This study suggests that controlling blood sugar levels can facilitate better self-control.

Low glucose diets are not individually designed and generally are not sustained: There are diets that are aimed at controlling the level of glucose consumed but they are designed for the generic consumer and cannot take into account the individual's unique ability to digest foods. Moreover, the ability to stay on a diet over the long term has shown to be difficult for most people. A study conducted by Salem College, University of South Carolina and Cornell University in 2011 and titled ‘Electronic feedback on a diet- and physical activity based lifestyle intervention for weight loss: a randomized controlled trial” concluded “Continuous self-monitoring from wearable technology with real time feedback may be particularly useful to enhance lifestyle changes that promote weight loss in sedentary overweight or obese adults.”

### Solution to the Problem

This invention calls for the use of Google's smart contact lens by non-diabetics to view their blood glucose level so as to be able to follow a diet unique to each individual that will maintain their glucose level at an optimum level on a continuous basis.

### Advantageous Effects of the Invention

Avoiding the normal peaks and valleys in the non-diabetic's blood glucose level will result in improving how the brain functions during the day. Memory, attentiveness, self-control, decision making, creativity, verbal and auditory reaction time and reading skills will all be enhanced to varying degrees. This is an application of the lens that is not currently considered by Google and is therefore an application that can be patented.

The ability to stay on a diet over the long term is enhanced when there is continuous feedback as to the progress being made. The lens will provide the wearer with updated readings every second. The non-diabetic will receive this information and will have the needed reinforcement to stay on the diet. Again, this is an application of the lens that is not currently considered by Google and is therefore an application that can be patented.

I claim:

1. The use of Google's smart contact lens for non-diabetics, those who do not have diabetes.

2. The use of Google's/Novartis/Alcon's smart contact lens for use by non-diabetics to measure changes in their blood glucose level resulting from their consumption of food and drink.

3. The use of Google's smart contact lens for use by non-diabetics to facilitate modifying their diet to control their blood glucose level so as to enhance a range of executive and non-executive functions including their memory, attentiveness, creativity, decision making skills, self-control, visual and auditory reaction time, psychomotor functions, verbal fluency and reading skills.

4-15. (canceled)
